(19) United States
(12) Patent Application Publication
(10) Pub. No.: US 2023/0139076 A1
(43) Pub. Date: May 4, 2023

(54) METHOD FOR PREDICTING MITOCHONDRIAL DNA MUTATION THRESHOLD, FERTILITY RISK AND OOCYTE RETRIEVAL NUMBER

(71) Applicant: The First Affiliated Hospital of Anhui Medical University Ltd., Hefei City (CN)

(72) Inventors: Yunxia Cao, Hefei City (CN); Dongmei Ji, Hefei City (CN); Yinan Du, Hefei City (CN); Ning Zhang, Hefei City (CN); Zhikang Zhang, Hefei City (CN); Zhuoli Liu, Hefei City (CN); Weiwei Zou, Hefei City (CN); Kai Zong, Hefei City (CN)

(21) Appl. No.: 17/541,457

(22) Filed: Dec. 3, 2021

(30) Foreign Application Priority Data
Nov. 3, 2021 (CN) 202111293327.9

(51) Int. Cl. G16B 50/30; G06F 17/18; G16H 50/30

(52) U.S. Cl. CPC G16B 50/30; G06F 17/18; G16H 50/30

(57) ABSTRACT

A method for predicting mtDNA mutation threshold, fertility risk and oocyte retrieval number includes: establishing an incidence probability prediction model of mitochondrial mutation and estimating a mutation threshold s; establishing a fertility risk prediction model and predicting a fertility risk of a mutation carrier; and establishing an oocyte retrieval prediction model by binomial distribution and calculating oocyte retrieval number by PGT of a mutation carrier. The incidence probability prediction model is used to estimate a threshold value of common mtDNA mutations and predict a mother's fertility risk and the oocyte retrieval number by PGT. On the basis of the fertility risk prediction model and oocyte retrieval prediction model, the fertility risk and a minimum oocyte retrieval number by PGT needed to give birth to a healthy offspring can be calculated by knowing a mitochondrial DNA mutation ratio of mutation carrier which is helpful for genetic management and genetic consultation.

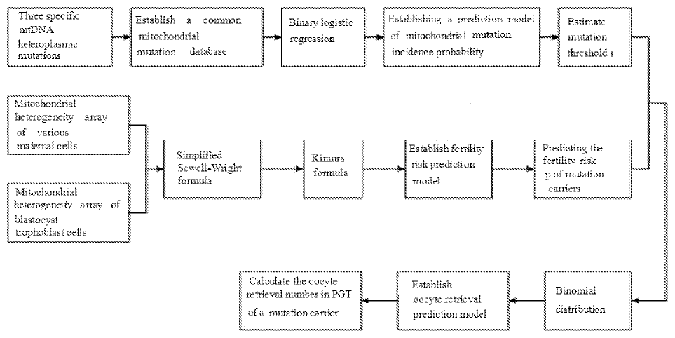

### TECHNICAL FIELD

The invention relates to the technical field of mitochondrial disease prediction, in particular to a method for predicting mitochondrial DNA mutation threshold, fertility risk and oocyte retrieval number.

### BACKGROUND

The mitochondrial disease is one of the most common and serious genetic diseases, and has an estimated incidence of 1:4300. Mitochondrial DNA (mtDNA) mutation is a common cause of mitochondrial diseases. Due to the lack of protective histone and imperfect DNA repair mechanism, the mutation rate of mtDNA is higher than that of nuclear DNA. Since mitochondria can contain multiple DNA copies, the mtDNA mutation can affect all gene copies (called homoplasmy) or some copies (called heteroplasmy) at the same time. Homoplasmic mutations are usually relatively mild and affect only one organ or tissue while heteroplasmic mutations can affect multiple organ systems and cause severe systemic mitochondrial genetic diseases. At present, it is estimated that about 0.5% of the global population carries pathogenic mtDNA mutations, and more than 300 pathogenic mtDNA mutations have been reported. However, there is no effective treatment for mitochondrial mutation. Because mtDNA mutation is only transmitted from mother to offspring, developing methods to prevent maternal transmission is a high priority.

A human oocyte contains about 100,000 mtDNA molecules. Due to the genetic bottleneck of mitochondria, only a small amount of mtDNA can be transmitted during oogenesis, which leads to considerable differences in the mutation level of offspring.

Mutation level(the ratio of mutation to normal mtDNA) is related to the occurrence of clinical symptoms and the severity of diseases. The bottleneck means that for women carrying mtDNA mutations, the mtDNA mutation level of the offspring is uncertain and the onset of mtDNA disease is unpredictable. Preimplantation genetic testing (PGT) can detect the level of mtDNA mutation in early embryos and select the best embryo for transplantation, so as to prevent the onset of the next generation. PGT has been widely used to prevent maternal transmission of mtDNA mutation. However, the standard operating standard of PGT has not been established. At present, it is not clear how to select suitable oocytes for transplantation and how many oocytes need to be obtained at most before transplanting suitable embryos. Therefore, the invention proposes a method for predicting mitochondrial DNA mutation threshold, fertility risk and the oocyte retrieval number to solve the problems existing in the prior art.

### SUMMARY

In view of the above problems, the purpose of the invention is to propose a method for predicting mitochondrial DNA mutation threshold, fertility risk and oocyte retrieval number. The method uses an incidence probability prediction model to estimate the threshold of common mtDNA mutations and predict mother's fertility risk and oocyte retrieval number by PGT. On the basis of the established fertility risk prediction model and oocyte retrieval prediction model, it is only necessary to know the mitochondrial DNA mutation ratio of a mutation carrier to calculate the fertility risk and the minimum oocyte retrieval number by PGT needed to give birth to a healthy offspring.

In order to achieve the purpose of the invention, the invention is realized by the following technical scheme: the method for predicting mitochondrial DNA mutation threshold, fertility risk and oocyte retrieval number comprises the following steps:

step 1: based on three specific mtDNA heteroplasmic mutations, m.8993T>G, m.8344A>G and m.3243A>G, a mitochondrial pedigree database is established as a common mitochondrial mutation database, and then based on the mutation level, binary logistic regression is adopted to predict the incidence probability, and an incidence probability prediction model of mitochondrial mutation is established, and then the mutation threshold s is estimated by using the incidence probability prediction model;

step 2: based on the mitochondrial heteroplasmy array of various maternal cells and the mitochondrial heteroplasmy array of blastocyst trophoblast cells, firstly, a distribution model of mtDNA mutation level of offspring, namely a fertility risk prediction model, is established by adopting a simplified Sewell-Wright formula and Kimura formula, and then, according to the estimated mutation threshold s and the distribution model of mtDNA mutation level of offspring, the cumulative probability affected in mtDNA distribution, namely fertility risk p, is calculated from 0% to mutation threshold s;

step 3: assuming that oocytes to be taken are X in number/quantity, so as to ensure that the probability of mutation levels of at least A number of embryos being lower than mutation threshold s is greater than 95%, and the proportion of fertilized eggs developing into normal embryos is k, based on the fertility risk and assumed conditions, establishing an oocyte retrieval prediction model by using binomial distribution and calculating the oocyte retrieval number by PGT of a mutation carrier through the oocyte retrieval prediction model;

step 4: first, get the universal mutation threshold and parameter b by fitting the reported/known mutation data, then use the universal mutation threshold and parameter b to establish universal fertility risk prediction model and PGT oocyte retrieval prediction model, and then use the universal prediction models to predict the fertility risk and oocyte retrieval number by PGT of a mutation carrier when the mutation level of the mutation carrier is known.

The further improvement is that in step 1, families in mitochondrial pedigree database are classified into three types: “familial”, “de novo” and “uninformative”, and family history data in mitochondrial pedigree database come from Mitomap and hospitals, and if the family history is positive, the family is “familial”; if the family history is negative, and the mtDNA mutation levels of the proband's mother and all tested maternal relatives are 0%, then the family is “de novo”, and the rest families are considered “uninformative” due to insufficient information.

The further improvement is that in step 1, familial pedigrees in the mitochondrial pedigree database are included in analysis; and specifically mean values of mtDNA mutation levels in blood and muscle are taken for analysis, wherein blood data of m.3243A>G mutation is age-corrected, as shown in the following formula:

Age−corrected blood mutation level=(blood mutation level)/0.977^{(age+12)};

where only the age-corrected mutation level of less than 95% is included in the analysis to avoid over-correction; considering the limitation of detection sensitivity, when a mother is a carrier of mtDNA mutation with clinical symptoms, an offspring with detected mtDNA mutation level of 0% is also included in the analysis, which is marked as 1%.

The further improvement is that in step 1, the incidence probability prediction model of mitochondrial mutation is:

$$Ln \frac{y}{1 - y} = {β}_{0} + {β}_{1} x ;$$

the estimation of mutation threshold s specifically is as follows: using the incidence probability prediction model of mitochondrial mutation, taking a non-morbidity probability of over 95% as the cut-off point, and determining the value of corresponding mtDNA mutation level as the threshold value of corresponding mtDNA mutation, and the embryo with mutation level lower than the threshold value is a transferable “safe embryo”.

The further improvement is that in step 2, the simplified Sewell-Wright formula is as follows:

V=p₀(1−p₀)[1−e^{-t/N}^{_{eff}}]=p₀(1−p₀)(1−b),

b=e^{-t/N}^{_{eff}};

where the simplified Sewell-Wright formula is a function of four parameters p₀, t, N_{eff} and V; p₀ is the original mtDNA mutation level, t is number of generation, N_{eff} is the effective population size, and V is the variance of mtDNA mutation level of multiple maternal oocytes.

The further improvement is that the Kimura formula is as follows:

f(0)=(1−p₀)+Σᵢ₌₁^{∞}(2i+1)p₀(1−p₀)(−1)ⁱF(1−i,i+2,2,1−p₀)b^{i(i+1)/2},

Ø(x)=Σᵢ₌₁^{∞}i(i+1)(2i+1)p₀(1−p₀)F(1−i,i+2,2,x)Fⁱ(1−i,i+2,2,p₀)b^{i(i+1)/2},

f(1)=p₀+Σᵢ₌₁^{∞}(2i+1)p₀(1−p₀)(−1)ⁱF(1−i,i+2,2,p₀)b^{i(i+1)/2};

p₀ and V are substituted into the formula for calculating b, and b is substituted into the Kimura formula to calculate the distribution of mtDNA mutation level.

The further improvement is that in step 3, the oocyte retrieval prediction model is as follows:

Σᵢ₌₀^{A-1}Cₖₓⁱpⁱ(1−p)^{kX-i}<0.05;

when A=1, the oocyte retrieval prediction model is simplified as:

$$X > \frac{{log}_{1 - p} 0.05}{k} ;$$

Where X is the predicted oocyte retrieval number.

The method has the following beneficial effects: the incidence probability prediction model is used to estimate the threshold value of common mtDNA mutations, and predict the mother's fertility risk and oocyte retrieval number; on the basis of the established fertility risk prediction model and oocyte retrieval prediction model, the fertility risk and the minimum oocyte retrieval number by PGT needed to give birth to a healthy offspring can be calculated only by knowing the mitochondrial DNA mutation ratio of a mutation carrier, which is helpful for clinicians to carry out genetic management and provide genetic consultation for families carrying mtDNA heteroplasmic mutations, and provide PGT standard guide, and can help parents decide whether they should have PGT at the same time.

### BRIEF DESCRIPTION OF THE FIGURE

In order to explain the embodiments of the invention or the technical scheme in the prior art more clearly, the figure used in the embodiments or the description of the prior art will be briefly introduced below. Apparently, the figure in the following description is only some embodiments of the invention, and other figures can be obtained according to the figure for those of ordinary skill in the art without paying creative labor.

The figure is a schematic flow chart of a method according to an embodiment of the invention.

### DETAILED DESCRIPTION OF EMBODIMENTS

The following will clearly and completely describe the technical scheme in the embodiments of the invention with reference to the figure in the embodiments of the invention. Apparently, the described embodiments are only part of the embodiments of the invention, not all of them. Based on the embodiments of the invention, all other embodiments obtained by those of ordinary skill in the art without creative labor fall in the scope of protection of the invention.

In the description of the invention, it should be noted that the orientation or position relationships indicated by the terms “center”, “upper”, “lower”, “left”, “right”, “vertical”, “horizontal”, “inner” and “outer” are based on the orientation or position relationships shown in the figure, which are only for the convenience of describing the invention and simplifying the description, rather than indicating or implying it. In addition, the terms “first”, “second”, “third”, “fourth”, etc. are only used for descriptive purposes and cannot be understood as indicating or implying relative importance.

In the description of the invention, it should be noted that unless otherwise specified and limited, the terms “install”, “connect” and “communicate” should be understood in a broad sense, for example, fixed connection, detachable connection or integrated connection, or connected mechanically or electrically; or directly connected, indirectly connected through an intermediate medium; or communicated inside two elements. For those of ordinary skill in the art, the specific meanings of the above terms in the invention can be understood under specific circumstances.

Referring to the figure, this embodiment provides a method for predicting mitochondrial DNA mutation threshold, fertility risk and oocyte retrieval number, which includes the following steps:

step 1, firstly, based on three specific mtDNA heteroplasmic mutations, m.8993T>G, m.8344A>G and m.3243A>G, establishing a mitochondrial pedigree database as a common mitochondrial mutation database, and then based on mutation level, predicting the incidence probability using binary logistic regression, and establishing a prediction model of mitochondrial mutation incidence probability. With more than 95% probability of disease-free as the cut-off point, the value of corresponding mtDNA mutation level is determined as the threshold value of corresponding mtDNA mutation, and embryos with mutation level lower than the threshold value are transferable “safe embryos”, and the prediction model of mitochondrial mutation incidence probability is as follows:

$$Ln \frac{y}{1 - y} = {β}_{0} + {β}_{1} x ;$$

Families in mitochondrial pedigree database are classified into three types: “familial”, “de novo” and “uninformative”, and family history data in mitochondrial pedigree database come from Mitomap (https://www.mitomap.org/MITOMAP) and hospitals, and if the family history is positive, the family is “familial”; if the family history is negative, and the mtDNA mutation level of the proband's mother and all tested maternal relatives are 0%, then the family is “de novo”, and the rest families are considered “uninformative” due to insufficient information;

familial family in mitochondrial pedigree database is included in the analysis; and specifically the mean values of mtDNA mutation level in blood and muscle are taken for analysis, wherein the blood data of m.3243A>G mutation is age-corrected, as shown in the following formula:

Age−corrected blood mutation level=(blood mutation level)/0.977^{(age+12)};

where only the age-corrected blood mutation level of less than 95% is included in the analysis to avoid over-correction; considering the limitation of detection sensitivity, when the mother is a carrier of mtDNA mutation with clinical symptoms, the offspring with detected mtDNA mutation level of 0% are also included in the analysis, which is marked as 1%;

The threshold values of three specific mtDNA heteroplasmic mutations, m.8993T>G, m.8344A>G and m.3243A>G, are estimated respectively, as follows:

using m.8993T>G database, the prior probability of the disease is calculated to be 0.56, and the prediction model of the disease probability is established; the parameter 0 is calculated to be −5.451 (95% CI-5.816- −5.097), the parameter 1 is calculated to be 8.395 (95% CI7.920−8.885), and the prediction model equation is Lny/(1−y)=−5.451+8.395x. The area under the ROC curve of the model is 0.847 (95% CI0.834˜0.860), which indicates that the model fits well, and using this model, it is estimated that the threshold of m.8993T>G mutation is 29.86%;

using m.8993T>G database, the prior probability of the disease is calculated as 0.3, and the prediction model of the disease probability is established. Similarly, parameter 0 is calculated as −3.827 (95% CI-4.006- −3.654), and parameter 1 is calculated as 5.463 (95% CI 5.205−5.728), and the prediction model equation is Lny/(1−y)=−3.827+5.463x. The area under the ROC curve of the model is 0.867 (95% CI 0.859−0.876), and using this model, it is estimated that the threshold of m.8344A>G mutation is 16.15%;

using m.8993T>G database, the prior probability of the disease is calculated to be 0.54, and the prediction model of the disease probability is established. The parameter 0 is calculated to be −1.696 (95% CI-1.806- −1.588), the parameter 1 is calculated to be 4.213 (95% CI 4.027−4.402), and the prediction model equation is Ln y/(1−y)=−1.696+4.213x; the area under the ROC curve of the model is 0.761(95% CI0.751−0.770).

step 2, based on the mitochondrial heteroplasmy array of various maternal cells and the mitochondrial heteroplasmy array of blastocyst trophoblast cells, firstly, a distribution model of offspring mtDNA mutation level, namely, a fertility risk prediction model, is established by using simplified Sewell-Wright formula and Kimura formula, and then according to the estimated mutation threshold s and the distribution model of offspring mtDNA mutation level, the cumulative probability of being affected in mtDNA distribution between 0% and mutation threshold s is calculated, and the cumulative probability is fertility risk p. The simplified Sewell-Wright formula is as follows:

V=p₀(1−p₀)[1−e^{-t/N}^{_{eff}}]=p₀(1−p₀)(1−b),

b=e^{-t/N}^{_{eff}};

Kimura's formula is as follows:

f(0)=(1−p₀)+Σᵢ₌₁^{∞}(2i+1)p₀(1−p₀)(−1)ⁱF(1−i,i+2,2,1−p₀)b^{i(i+1)/2},

Ø(x)=Σᵢ₌₁^{∞}i(i+1)(2i+1)p₀(1−p₀)F(1−i,i+2,2,x)Fⁱ(1−i,i+2,2,p₀)b^{i(i+1)/2},

f(1)=p₀+Σᵢ₌₁^{∞}(2i+1)p₀(1−p₀)(−1)ⁱF(1−i,i+2,2,p₀)b^{i(i+1)/2};

where, the simplified Sewell-Wright formula is a function of four parameters p₀, t, N_{eff} and V; p₀ is the original mtDNA mutation level, t is algebra, N_{eff} is the effective population size, and V is the variance of mtDNA mutation level of multiple maternal oocytes. p₀ and V are substituted into the formula for calculating b, and b is substituted into Kimura equation to calculate the distribution of mtDNA mutation level;

step 3: assume that the oocyte to be taken is X, so as to ensure that the probability of mutation level of at least A embryos being lower than mutation threshold s is greater than 95%, and the proportion of fertilized eggs developing into normal embryos is K. Based on the fertility risk and assumed conditions, a prediction model of oocyte retrieval is established by using binomial distribution, that is, an oocyte retrieval prediction model, and the oocyte retrieval number by PGT of a mutation carrier is calculated by the oocyte retrieval prediction model. The prediction model of oocyte retrieval is as follows:

Σᵢ₌₀^{A-1}Cₖₓⁱpⁱ(1−p)^{kX-i}<0.05;

when A=1, the model is simplified as:

$$X > \frac{{log}_{1 - p} 0.05}{k} ;$$

where X is the predicted oocyte retrieval number;

step 4: first, get the universal mutation threshold and parameter b by fitting the reported/known mutation data, then use the universal mutation threshold and parameter b to establish universal fertility risk prediction model and oocyte retrieval prediction model, and then use the universal prediction models to predict the fertility risk and oocyte retrieval number by PGT of a mutation carrier when the mutation level of the mutation carrier is known.

The above is only preferred embodiments of the invention, and is not intended to limit the invention. Any modifications, equivalent substitutions, improvements, etc. made within the spirit and principles of the invention shall fall in the scope of protection of the invention.

What is claimed is:

1. A method for predicting mitochondrial deoxyribo nucleic acid (mtDNA) mutation threshold, fertility risk and oocyte retrieval number, comprising:
step 1, establishing a mitochondrial pedigree database based on three specific mtDNA heteroplasmic mutations of m.8993T>G, m.8344A>G and m.3243A>G as a common mitochondrial mutation database, predicting an incidence probability by binary logistic regression based on mutation levels and establishing an incidence probability prediction model of mitochondrial mutation, and estimating a mutation threshold s by using the incidence probability prediction model;
step 2, establishing a distribution model of mtDNA mutation level of offsring, namely a fertility risk prediction model, based on a mitochondrial heteroplasmy array of various maternal cells and a mitochondrial heteroplasmy array of blastocyst trophoblast cells by adopting simplified Sewell-Wright formula and Kimura formula, and calculating a cumulative probability affected in a mtDNA distribution from 0% to the mutation threshold s according to the estimated mutation threshold s and the distribution model of mtDNA mutation level of offspring, wherein the cumulative probability namely is fertility risk p;
step 3, assuming that oocytes to be taken are X to thereby ensure that a probability of mutation levels of at least A number of embryos being lower than the mutation threshold s is greater than 95%, and a proportion of fertilized eggs developing into normal embryos is k; establishing an oocyte retrieval prediction model, namely an oocyte retrieval prediction model, by using binomial distribution based on the fertility risk and conditions of the assuming, and calculating an oocyte retrieval number by preimplantation genetic testing (PGT) of a mutation carrier through the oocyte retrieval prediction model;
step 4: getting universal mutation threshold and parameter b by fitting known mutation data, using the universal mutation threshold and parameter b to establish universal fertility risk prediction model and PGT oocyte retrieval prediction model, and using the universal fertility risk prediction model and PGT oocyte retrieval prediction model to predict a fertility risk and an oocyte retrieval number by PGT of a mutation carrier when a mutation level of the mutation carrier is known.

2. The method for predicting mtDNA mutation threshold, fertility risk and oocyte retrieval number according to claim 1, wherein in step 1, families in the mitochondrial pedigree database are classified into three types: “familial”, “de novo” and “uninformative”, and family history data in the mitochondrial pedigree database come from Mitomap and hospitals, and if a family history is positive, the family is “familial”; if a family history is negative, and a mtDNA mutation level of a proband's mother and mtDNA mutation levels of all tested maternal relatives are 0%, then the family is “de novo”; and the rest of the families are considered as “uninformative” due to insufficient information.

3. The method for predicting mtDNA mutation threshold, fertility risk and oocyte retrieval number according to claim 1, wherein in step 1, familial pedigrees in the mitochondrial pedigree database are included in analysis; specifically, mean values of mtDNA mutation level in blood and muscle are taken for the analysis, of which blood data of m.3243A>G mutation is age-corrected according to the following formula:
Age−corrected blood mutation level=(blood mutation level)/0.977^{(age+12)};
where only the age-corrected blood mutation level of less than 95% is included in the analysis to avoid over-correction; considering a limitation of detection sensitivity, when a mother is a carrier of mtDNA mutation with clinical symptoms, an offspring with detected mtDNA mutation level of 0% is also included in the analysis and marked as 1%.

4. The method for predicting mtDNA mutation threshold, fertility risk and oocyte retrieval number according to claim 1, wherein in step 1, the incidence probability prediction model of mitochondrial mutation is:
$$Ln \frac{y}{1 - y} = {β}_{0} + {β}_{1} x ;$$
the estimating of the mutation threshold s specifically is as follows: using the incidence probability prediction model of mitochondrial mutation, taking a non-morbidity probability of over 95% as a cut-off point, and determining a value of corresponding mtDNA mutation level as a threshold value of corresponding mtDNA mutation, and an embryo with a mutation level lower than the threshold value is a transferable “safe embryo”.

5. The method for predicting mtDNA mutation threshold, fertility risk and oocyte retrieval number according to claim 1, wherein in step 2, the simplified Sewell-Wright formula is as follows:
V=p₀(1−p₀)[1−e^{-t/N}^{_{eff}}]=p₀(1−p₀)(1−b),
b=e^{-t/N}^{_{eff}};
the simplified Sewell-Wright formula is a function of four parameters p₀, t, N_{eff} and V, p₀ is an original mtDNA mutation level, t is number of generation, N_{eff} is an effective population size, and V is a variance of mtDNA mutation level of multiple maternal oocytes.

6. The method for predicting mtDNA mutation threshold, fertility risk and oocyte retrieval number according to claim 5, wherein the Kimura formula is as follows:
f(0)=(1−p₀)+Σᵢ₌₁^{∞}(2i+1)p₀(1−p₀)(−1)ⁱF(1−i,i+2,2,1−p₀)b^{i(i+1)/2},
Ø(x)=Σᵢ₌₁^{∞}i(i+1)(2i+1)p₀(1−p₀)F(1−i,i+2,2,x)Fⁱ(1−i,i+2,2,p₀)b^{i(i+1)/2},
f(1)=p₀+Σᵢ₌₁^{∞}(2i+1)p₀(1−p₀)(−1)ⁱF(1−i,i+2,2,p₀)b^{i(i+1)/2};
p₀ and V are substituted into the formula for calculating b, and b is substituted into the Kimura formula to calculate a distribution of mtDNA mutation level.

7. The method for predicting mtDNA mutation threshold, fertility risk and oocyte retrieval number according to claim 1, wherein in step 3, the oocyte retrieval prediction model is as follows:
Σᵢ₌₀^{A-1}Cₖₓⁱpⁱ(1−p)^{kX-i}<0.05;
when A=1, the oocyte retrieval prediction model is simplified as:
$$X > \frac{{log}_{1 - p} 0.05}{k} ;$$
where X is predicted oocyte retrieval number.
